(19) United States
(12) Patent Application Publication
(10) Pub. No.: US 2003/0097113 A1
(43) Pub. Date: May 22, 2003

(54) Absorbent product with reduced rewet properties under load

(76) Inventor: Kenneth John Molee, Suwanee, GA (US)

Correspondence Address: Christopher C. Campbell, Esq., Hunton & Williams, 1900 K Street, NW, Suite 1200, Washington, DC, 20006-1109, US

(21) Appl. No.: 09/970,673

(22) Filed: Oct. 5, 2001

(51) Int. Cl. A61F 13/15; A61F 13/20

(52) U.S. Cl. 604/385.101; 604/378; 604/383

(57) ABSTRACT

An absorbent article having a substantially liquid impervious outer layer, a substantially liquid pervious inner layer overlaying and associated with the outer layer, an absorbent core disposed between the outer layer and inner layer, and an apertured film disposed between the inner layer and the absorbent core. The apertured film has a liquid impervious film surface with a plurality of protrusions extending therefrom towards the absorbent core. Each protrusion terminates at an aperture in the apertured film. The absorbent article has a 200 milliliter rewet under load of less than about 1.25 grams and a 300 milliliter rewet under load of less than about 4 grams

### BACKGROUND OF INVENTION

1. Field of the Invention

The invention relates to the field of absorbent products and more particularly to an absorbent product having substantially reduced rewetting when the core is at or near the saturation point and placed under load.

2. Description of Related Art

Traditionally absorbent garments have included a liquid permeable inner layer (often referred to as a “topsheet”) facing the user, an absorbent core for absorbing and storing body exudates, and a liquid impermeable outer layer (often referred to as a “backsheet”) for containing body exudates within the article. Such articles are often used multiple times before being removed. For example, an infant may urinate into a diaper several times before the diaper is removed and replaced. Under such circumstances, it is desirable for the article to continue to be comfortable to the user after the first use and any subsequent uses.

One factor contributing to the comfort of an article intended for multiple uses is the article's rewet characteristics. Rewet is caused when fluid is released from an absorbent core of the article and migrates to the body-contacting surface of the article. When the fluid contacts the user's skin, it can cause discomfort and may cause medical problems, such as irritation, rashes, and infections.

Rewet has many causes. Typically, rewet occurs when the absorbent core of the article becomes saturated and unable to hold more fluid. Rewet also occurs when the absorbent core is below its saturation point. For example, rewet may occur when the absorbent core can not absorb a given volume of fluid quickly enough. Rewet also may occur when an absorbent article is placed under pressure, causing fluid to be mechanically forced out of the absorbent core. Typically, when an absorbent article is at a high saturation level or at the saturation point, rewet becomes an increased problem, as the compressive force required to force fluid out of the absorbent core generally decreases as the amount of saturation increases.

Attempts have been made to reduce the rewet characteristics of absorbent articles. One suggested solution is to provide an acquisition or transport layer between the absorbent core and the liquid permeable body contacting layer. The acquisition layer distributes fluids across the surface of the absorbent core to improve the absorbent core's ability to rapidly absorb fluids. Once the fluids are absorbed by the core, the acquisition layer acts as a relatively dry boundary between the inner layer and the absorbent core to prevent rewet. This benefit is minimal, however, because conventional acquisition layers do not prevent fluid from reemerging from the core when the core is placed under pressure, especially when the core is at or near the saturation point, and rewet may occur under these circumstances.

Another suggested solution is to provide an apertured film layer to prevent backflow of fluid out of the absorbent core. Apertured films having three-dimensional funnels have been found to provide preferential fluid flow away from the surface of the film and through the funnels. Such films may provide beneficial rewet capabilities when properly employed in the construction of an absorbent article. U.S. Pat. No. 6,171,291 issued to Palumbo et al., discloses the use of an apertured film body-contacting inner layer to prevent rewet. It has been recognized, however, that the use of an apertured film as an inner layer may necessitate careful selection of, or additional modifications to, the apertured film to provide it with a suitable feel and texture for use as a body-contacting surface. These and other considerations may increase the cost of the article or reduce design flexibility.

An apertured layer may also be used as a rewet barrier located between the inner layer and the absorbent core. U.S. Pat. No. 5,603,707 issued to Trombetta, et al., discloses an absorbent article having a macroscopically expanded apertured web acting as a rewet barrier disposed between the absorbent core and an additional fibrous acquisition layer located adjacent the inner layer. U.S. Pat. No. 4,323,069 issued to Ahr et al., also discloses an intermediate layer comprising capillary openings, located between a fibrous acquisition layer and an absorbent core. U.S. Pat. No. 5,352,217 issued to Curro, discloses the use of multiple layers of apertured film inner layers to reduce rewet.

Despite the above disclosures and other attempts to prevent or reduce rewet, the problem of rewet remains, particularly when an absorbent article is at or near the saturation point. In view of the foregoing considerations, there is a continuing need to provide absorbent articles with improved rewet characteristics. More specifically, there is a need to provide absorbent articles providing improved rewet prevention at high saturation levels.

### SUMMARY OF THE INVENTION

It is an object of the present invention to provide an absorbent article having improved rewet prevention at high saturations levels. It is a further object of the present invention to provide an absorbent article that has improved comfort. It is yet a further object of the present invention to provide an absorbent garment that provides improved resistance to rashes, irritation and infections.

These and other objects of the invention may be provided by an absorbent article having a liquid impervious outer layer, a liquid pervious inner layer overlaying and attached to the outer layer, an absorbent core disposed between the outer layer and inner layer, and an apertured film disposed between the inner layer and the absorbent core. The apertured film has a liquid impervious film surface and a plurality of protrusions extending from the surface towards the absorbent core. Each protrusion terminates at an aperture in the apertured film.

The absorbent article of the present invention has a 200 milliliter rewet under load of less than about 1.25 grams and a 300 milliliter rewet under load of less than about 4 grams. In other aspects of the invention, the absorbent article may have a 200 milliliter rewet under load of less than about 0.80 grams, or of about 0.56 grams. In still other aspects of the invention, the absorbent article may have a 300 milliliter rewet under load of less than about 3.00 grams, or of about 1.94 grams.

In another aspect of the present invention, a tissue layer may surround the absorbent core and the apertured film. In yet another aspect, a transfer layer may be disposed between the inner layer and the absorbent core.

In various aspects of the invention, the apertured film may cover substantially all of the surface of the absorbent core that faces the inner layer, or may only cover an insult region of the absorbent core.

In other aspects of the invention, the protrusions may extend in a substantially orthogonal direction from the liquid impermeable surface, and may have hexagonal, circular, or slit shapes. The area of each protrusion may be less at the aperture than at the film surface.

In various other aspects of the invention, the apertured film may have a loft of: between about 0.500 millimeters and about 1.500 millimeters, between about 0.750 millimeters and about 1.250 millimeters, or about 1.000 millimeters.

In still other aspects of the invention, the apertured film may have a porosity of: between about 71.5 m³ₐᵢᵣ/min m²_{film} and about 122 m³ₐᵢᵣ/min m²_{film}, between about 84.0 m³ₐᵢᵣ/min m²_{film} and about 109 m³ₐᵢᵣ/min m²_{film}, and about 96.5 m³ₐᵢᵣ/min m²_{film}.

In further aspects of the invention, the apertured film may have a drain rate of: between about 597 kg/s m²_{film} and about 995 kg/s m²_{film}, between about 697 kg/s m²_{film} and about 896 kg/s m²_{film}, or about 796 kg/s m²_{film}.

### BRIEF DESCRIPTION OF THE DRAWINGS

The embodiments of the present invention may be better understood with reference to the accompanying drawings, in which:

FIG. 1 is a partially cut away view of an embodiment of the present invention depicted in the laid-flat position;

FIG. 2 is a cut away view of an embodiment of an apertured film of the present invention;

FIG. 3 is a drawing of a test assembly for measuring rewet under load;

FIG. 4a is a top view of a strikethrough plate;

FIG. 4b is a cut away view of the strikethrough plate of FIG. 4a, shown along reference line AA;

FIG. 5 is a drawing of a surface dryness measuring equipment test device; and

FIG. 6 is a graph showing surface wetness test results.

### DETAILED DESCRIPTION OF THE INVENTION

As used herein, the terms “absorbent article,” “article,” “absorbent garment” and “garment” refer to items that absorb and contain fluid discharges and exudates, and more specifically refer to articles that are placed against or in proximity to the body of the wearer to absorb and contain various bodily discharges. A non-exhaustive list of examples of absorbent articles and absorbent garments includes diapers, diaper cores, diaper covers, disposable diapers, training pants, feminine hygiene products, adult incontinence products, absorbent sheets, absorbent wipes, and the like. The claims are intended to cover all of the forgoing classes of absorbent articles, without limitation, whether disposable, unitary or otherwise. These classifications are used interchangeably throughout the specification, but are not intended to limit the claimed invention. The invention will be understood to encompass, without limitation, all classes of absorbent articles and garments, including those described above.

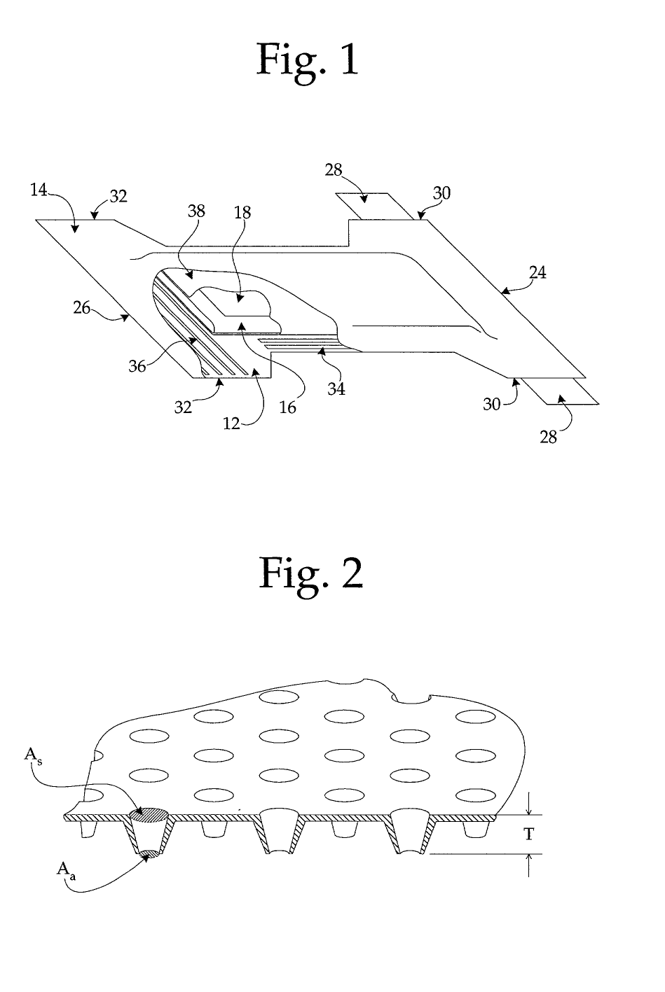

Referring now to FIGS. 1 and 2, an embodiment of the present invention generally comprises a garment 10 having an outer layer 12, overlaid by an inner layer 14. An absorbent core 16 for absorbing and retaining body exudates is disposed between the inner layer 14 and the outer layer 12. An apertured film 18 is disposed between the absorbent core 16 and the inner layer 14. The apertured film comprises a plurality of protrusions 20 that extend from the surface of the apertured film 18 towards the absorbent core 16. Each protrusion terminates at an aperture 22.

The garment 10 may be made in a variety of shapes and sizes, depending on the particular application for which it is designed. In one embodiment the garment 10 may be an infant diaper. In another embodiment, the garment 10 may be an adult incontinence product. In yet another embodiment, the garment 10 may be a feminine care product. Other uses of the invention with absorbent products are possible, and the invention is not to be limited by the recitation of these embodiments.

In the embodiment depicted in FIG. 1, the garment 10 is an infant diaper. In FIG. 1, the garment 10 is shown in the laid-flat position with any shirrs or wrinkles caused by elastics pulled flat. For demonstrative purposes, the embodiment of FIG. 1 is shown partially cut away. In such an embodiment, the garment may comprise separate first and second waist edges 24, 26 that, in use, are brought together to encircle a wearer's waist. The garment is held together by attachment tabs 28 located on a first pair of side edges 30 adjacent the first waist edge 24. The attachment tabs 28 attach to a portion of the garment 10 adjacent to the second waist edge 26. The attachment tabs may be any fastening systems known in the art, and may comprise, for example, a hook and loop fasteners, buttons, pins, adhesive tapes, and the like. When the attachment tabs 28 are attached, the first pair of side edges 30 are located proximal to a second pair of side edges 32, and the portion of the garment extending between the pairs of side edges 30, 32 forms a crotch region with leg holes for encircling the wearer's legs.

The garment 10 may also comprise various elastic elements, such as leg gathers 34 and waist elastics 36, to help prevent body exudates from leaking out of the garment 10. The general structural design and fabrication of diapers and other absorbent garments is generally known in the art.

The outer layer 12 is preferably made from a substantially liquid impervious material, and as a variety of such materials are known and available, the embodiments of the invention are not intended to be limited to any particular material. The selection and manufacture of such materials is well known in the art, and is disclosed, for example, in U.S. Pat. No. 6,123,694 issued to Peniak et al., and U.S. Pat. No. 6,176,952 issued to Maugans et al., each of which is incorporated herein by reference in its entirety, and in a manner consistent with the present invention. In one embodiment, the outer layer 12 is made from a thin thermoplastic material, such as a pigmented polyethylene film having a thickness in the range of 0.02-0.04 mm. The outer layer 12 may also have a laminate construction comprising one or more layers of meltblown polypropylene or meltblown polyethylene, sandwiched between layers of spun-bonded material (often referred to as an “SMS” laminate). The outer layer 12 may be a laminate of several layers, and additional layers may be added to the outer layer 12 in order to provide the outer layer 12 with other desirable properties, such as to improve the tactile feel, or “hand,” of the outer layer 12. The outer layer 12 may also be entirely or partly gas pervious to allow the garment to circulate air, or “breathe.” In one embodiment of the invention, the outer layer 12 has a multi-paneled construction in which the outer layer 12 comprises several pieces of material, which may have dissimilar physical properties, joined at or near their edges with little or no overlap.

The outer layer 12 may essentially define the outer perimeter of the garment 10, such that none of the other parts of the garment 10 (except, in some cases, for the attachment tabs 28) extend beyond the outline of the outer layer 12 when the garment 10 is laid flat, as is depicted in the embodiment of FIG. 1. However, in other embodiments the outer layer 12 may not define the outer perimeter of the garment 10, and other parts may extend beyond the edges of the outer layer 12.

The inner layer 14, which preferably overlays and is operatively associated with the outer layer 12, may comprise a substantially liquid pervious material to allow body exudates to penetrate into the absorbent core 16. A variety of suitable inner layer 14 materials are known in the art and commercially available, and the embodiments of the invention are not intended to be limited to any particular material. In a preferred embodiment, the inner layer 14 is a hydrophilic nonwoven material, such as a spunbonded polypropylene which may be thermally bonded by patterned calendar rolls. The inner layer 14 may also comprise a carded polyester fiber with a latex binder or any other suitable substantially liquid pervious material. The inner layer 14 may be treated over all or part of its surface to render it hydrophilic. The inner layer 14 may also be zone-treated with a surfactant to render it hydrophilic only in certain target areas. The inner layer 14 may also be treated with skin treating ingredients, such as aloe, vitamin E, and the like. The surface treatment can be accomplished by a variety of methods known in the art.

In one embodiment of the present invention, the inner layer 14 comprises a laminate of several layers of material, which may have different physical properties. In another embodiment, the inner layer 14 is made from several pieces of material joined at or near their edges with little or no overlap, which may have dissimilar physical properties (multi-panel construction). Such an embodiment is disclosed, for example, in U.S. Pat. No. 5,275,590 issued to Huffman et al., which is incorporated herein by reference in its entirety, and in a manner consistent with the present invention.

In the embodiment of the invention depicted in FIG. 1, the inner layer 14 has substantially the same planar dimensions as the outer layer 12, such that the perimeter of the inner layer 14 matches the perimeter of the outer layer 12. In other embodiments, the inner layer 14 may be larger or smaller than the outer layer 12, and may have a different general shape. The inner layer 14 is preferably large enough to completely cover all of the parts of the garment that are located between the outer layer 12 and the wearer, such as the leg gathers 34, absorbent core 16, and waist elastics 36.

The inner layer 14 and outer layer 12 are “operatively associated” with one another. As used herein, “operatively associated,” or simply “associated,” refers to any joining method that affixes one object to another object such that the two objects maintain their desired relative positions during the useful life of the invention. “Operatively associated” includes directly joining one object to another, joining objects through one or more intermediary objects, and physically capturing an object in place by joining surrounding objects to one another.

Preferably, the inner layer 14 and outer layer 12 comprise materials that may be easily associated with one another to form seals, such as by impulse, resistant, hot air, rotary band, crimp, side-weld, ultrasonic sealing, or any other suitable method. These seals may be shaped as, for example, fin-seals, lap-seals or gussets. The inner layer 14 and outer layer 12 may be associated with one another by bonding them in substantially all areas not having intermediately placed parts, such that some or all of the intermediately placed, or “sandwiched,” parts are physically captured between the inner layer 14 and outer layer 12, and thereby associated with the garment 10, but not bonded to the outer layer 12 or inner layer 14. In one embodiment, the inner layer 14 is operatively associated with the outer layer 12 around the perimeter of the inner layer 14.

An absorbent core 16 is preferably disposed between the outer layer 12 and the inner layer 14. The absorbent core 16 may be made from any absorbent material or materials known in the art. In one embodiment of the invention, the absorbent core 16 comprises wood fibers or other fibers such as chemical wood pulp, or any other suitable liquid absorbing material, such as commercially available fluff pulp or fluffed bleached kraft softwood pulp. In another embodiment of the invention, the absorbent core 16 comprises a combination of a porous fibrous web and super absorbent particles. Such absorbent cores are known in the art and are disclosed, for example, in U.S. Pat. No. 5,281,207 issued to Chmielewski et al., which is incorporated herein by reference in its entirety, and in a manner consistent with the present invention.

In one embodiment, the absorbent core 16 may be surrounded by a liquid pervious tissue wrap 38, or other material. The tissue wrap may provide the absorbent core with improved strength, facilitate manufacturing, help contain loose absorbent material, or provide other functions. Such a tissue wrap 38 may surround all or part of the absorbent core, and may also surround all or part of other parts of the garment 10, such as the apertured film 18. Liquid pervious tissue wraps are known in the art, and skilled artisans will be able to select an appropriate tissue wrap for use with the present invention without undue experimentation.

The absorbent core 16 is generally elongated in a lengthwise direction extending between the first and second waist edges 24, 26, and may have a width that extends substantially throughout the width of the garment 10. In the embodiment depicted in FIG. 1, the absorbent core 16 is substantially rectangular in shape, however, it may also have rounded ends or other shapes, such as an “I” shape or a “T” shape. The absorbent core 16 may also have channels, grooves, pockets or other structural features. In one embodiment, the absorbent core 16 has “zoned” absorbent capacity that locates greater absorbent capacity in certain regions of the absorbent core 16 to provide additional benefits to the garment 10.

Those skilled in the art will appreciate and recognize that the garment 10 may further comprise additional layers located between the respective layers or components. For example, the garment may further comprise one or more layers of material (not shown) to provide additional fluid redistribution capabilities to the garment, which are often referred to as an “acquisition layer” or a “transfer layer.” Such multiple layer absorbent cores are known in the art and one example is disclosed in U.S. Pat. No. 5,439,458 issued to Noel et al., which is incorporated herein by reference in its entirety, and in a manner consistent with the present invention.

An apertured film 18 is located between the inner layer 14 and the absorbent core 16. In a preferred embodiment of the invention the absorbent core 16 is surrounded by a tissue wrap 38 and the apertured film 18 is located directly on the surface of the tissue wrap 38. In other embodiments, the apertured film 18 may be located within the tissue wrap 38. The apertured film 18 may be sized to cover substantially all of the inner layer-facing surface of the absorbent core 16, or it may be sized to cover a specific region of the absorbent core 16. For example, a local apertured film 18 that is substantially smaller than the inner layer-facing surface of the absorbent core 16 may be employed locally in regions where rewet is most likely to occur. The regions of the absorbent core 16 that are likely to experience rewet are typically those regions beneath the article insult point or points (i.e., where the fluids or exudates initially strike the article) and the surrounding areas. Such regions are referred to herein as “insult regions.” The ideal size and shape for the local apertured film 18 may vary with the application, and a skilled artisan will be able to determine these variables without undue experimentation. The use of such local apertured films 18 may provide a manufacturing cost savings.

FIG. 2 is a cut away depiction of an apertured film 18 of the present invention. The apertured film 18 may comprise any suitable liquid impervious material that may be formed to have the properties described herein. In a preferred embodiment, the apertured film comprises Tredegar product number X-27373, available from Tredegar Film Products Corporation, a corporation headquartered in Richmond, Va. In one embodiment the apertured film 18 comprises a polyethylene or polypropylene material. Protrusions 20 formed in the apertured film material extend from the apertured film surface 40 and apertures 22 are formed at the end of each protrusion 22. The protrusions 20 and apertures 22 may be microfunnel-like structures that are not visible or barely visible to the unaided eye. In a preferred embodiment, the protrusions extend in a substantially orthogonal direction from the apertured film surface 40, however, they may extend at any angle from the apertured film surface, and they may extend through an arcuate path. Because the absorbent article may be subjected to substantial compressive forces during manufacture, shipping, storage, and use, the protrusions 20 preferably have the ability to elastically deform when compressed, such that they substantially reassume their original shape after being compressed.

The size, shape, and distribution frequency of the protrusions 20 and apertures 22 may be modified to provide specific benefits to the garment 10. In a preferred embodiment, the protrusions 20 and apertures are designed to provide a substantially unimpeded flow of fluid from the inner layer 14 to the absorbent core 16, while simultaneously impeding the opposite flow of fluid. In addition, the protrusions 20 and apertures 22 may be designed to transport fluids having particular characteristics, such as viscosity, density, mass and flow rate.

Generally, the protrusions 20 and apertures 22 may be made with any geometric or non-geometric shape. In one embodiment, the protrusions 20 and apertures 22 are circular. In another embodiment, the protrusions 20 and apertures 22 are hexagonal. In yet another embodiment, the protrusions 20 and apertures 22 are slits. In addition, the protrusions 20 may have a shape at the apertured film surface 40 surface that transitions into an aperture 22 having a different shape. For example, the protrusions 20 may be hexagonal in shape at the apertured film surface 40 and transition to a substantially circular shape at the aperture 22.

In a preferred embodiment of the present invention, the area Aₛ of each protrusion 20 at the apertured film surface 40 is greater than the area Aₐ of each corresponding aperture 22. That is, the passage through each of the protrusions 20 becomes smaller at the aperture 22.

The protrusions 20 extend away from the apertured film surface 40, thereby providing the apertured film 18 with loft T which is the uncompressed thickness of the apertured film 18. In a preferred embodiment of the present invention the apertured film has a loft T of between about 0.500 millimeters and about 1.500 millimeters. In a more preferred embodiment of the present invention the apertured film has a loft T of between about 0.750 millimeters and about 1.250 millimeters. In a most preferred embodiment of the present invention the apertured film has a loft T of about 1.000 millimeters. It has been found that the value of the loft T may generally correspond to the apertured film's 18 ability to contain fluid volumes within the protrusions 20, however higher loft values may cause additional problems. For example, protrusions 20 having relatively high loft values may tend to collapse or fold over more easily, thereby preventing fluid from passing through them. Such protrusions 20 may also be more easily torn or damaged, causing them to separate from the film surface 40.

The apertured film 18 is porous and allows gasses to pass therethrough. The porosity of the apertured film 18 may be measured according to known test methods, such as those described in American Society for Testing and Materials test method ASTM D-737. The apertured film 18 preferably has a porosity of between about 71.5 cubic meters per minute per square meter of film (m³ₐᵢᵣ/min m²_{film}) and about 122 m³ₐᵢᵣ/min m²_{film}, and more preferably between about 84.0 m³ₐᵢᵣ/min m²_{film} and about 109 m³ₐᵢᵣ/min m²_{film}, and most preferably about 96.5 m³ₐᵢᵣ/min m²_{film}.

The apertured film 18 allows substantial fluid passage from the film surface 40 and through the apertures 20 to the other side of the apertured film 18. In a preferred embodiment, the apertured film 18 has a drain rate of about 597 kilograms per second per square meter of film (kg/s m²_{film}) and about 995 kg/s m²_{film}, more preferably between about 697 kg/s m²_{film} and about 896 kg/s m²_{film}, and most preferably of about 796 kg/s m²_{film}.

Those skilled in the art are capable of determining or measuring the properties of the apertured film 18, including the loft, porosity and drain rate. In addition, a skilled artisan is capable of making an apertured film 18 having the preferred properties described herein.

It has been found that an absorbent article having the above described characteristics provides an unexpected and surprising level of rewet prevention when the absorbent core is under load and at or near the saturation point of the article compared to absorbent articles using conventional constructions. The following examples demonstrate the improved efficacy of the present invention with respect to rewet characteristics.

### EXAMPLE 1

### Rewet Under Load Test

An embodiment of the present invention (the test garment) and a garment having a conventional construction (the conventional garment) were tested to determine their relative ability to suppress rewet while under load.

The test garment for the present example comprised an absorbent garment in the form of a training pant. The garment had an absorbent core 16 comprising a composite of about 16 grams of craft pulp of cellulose fibers and 13 grams of super absorbent polymer (SAP), such as cross linked sodium salt of acrylic acid super absorbent polymer. The absorbent core had a composite basis weight of about 667 grams per square meter (gsm), a length of about 435 millimeters, a width of about 100 millimeters, and was wrapped in a tissue layer 38. An apertured film transfer layer 18, as described herein, overlayed the absorbent core 16. In the present tests, the apertured film was Tredegar X-27373 apertured film.

The topsheet 14 of the test garment comprises a spunbond polypropylene nonwoven sheet having a basis weight of 15.0 gsm, that is treated with a zoned surfactant to render the topsheet more hydrophilic. The backsheet 12 is a fluid-impervious polyethylene film.

The saturation value for both the test garments and the conventional garments was between about 570 grams and 621 grams of simulated urine. The saturation value was determined by saturating 6 sample test garments, each having a weight within one standard deviation of the mean weight of 100 test garment samples, and weighing the amount of fluid retained by the test garments. The same procedure was repeated to determine the saturation value for the conventional garments.

For the saturation analysis, simulated urine was prepared by the following method: (1), placing 10 grams of Triton X-100 (available from Rohm & Haas Company, headquartered in Philadelphia, Pa.) into a flask; (2), filling the flask with deionized water to the 1000 milliliter mark to create an X-100 Solution; (3), placing 100 grams of the X-100 Solution into a container; (4), adding 360 grams of Sodium Chloride (NaCl) to the container; and (5), filling the container with deionized water to the 40 liter mark to create the simulated urine. Each of the six sample garments was weighed in the dry state then fully submerged in a tank full of simulated urine for one hour. After one hour, the each sample was removed from the tank and hung by one end for ten minutes. After the ten minutes elapsed, each sample was weighed to determine the saturated weight. The saturation value for each sample was recorded as the difference between the saturated weight and the dry weight.

In preparation for the test, the side seams of the test garment were severed and the test garment was laid flat with the inner layer side facing upwards. Simulated urine was prepared as described above. In addition, first, second, and third quantities of filter paper (Fisher brand, cut to 2.5 inches by 2.5 inches) weighing approximately 18 grams, 72 grams, and 90 grams, respectively, were weighed to obtain their dry weight to within one hundredth of a gram.

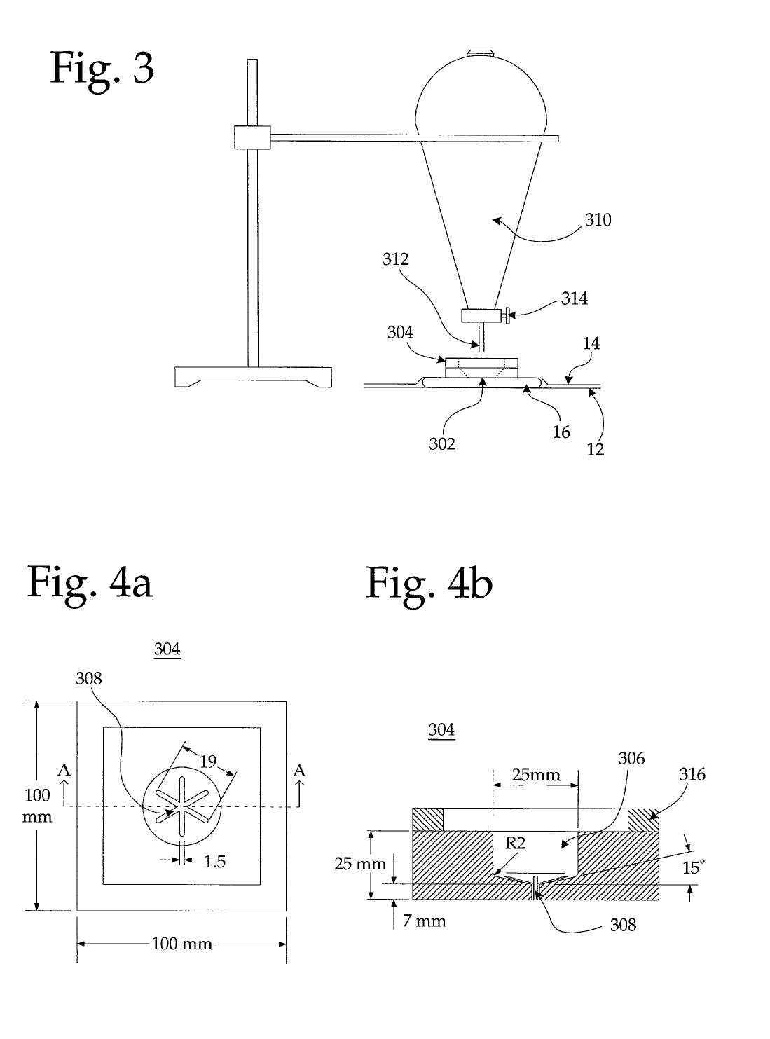

Referring now to FIGS. 3, 4a and 4b, the rewet test was performed using a strikethrough plate 304 to regulate the flow of synthetic fluid. The strikethrough plate 304 comprises an upper fluid cavity 306 and a lower strikethrough channel 308. The strikethrough channel 308 is placed in contact with the garment, and fluids poured into the strikethrough plate pass through the fluid cavity 306 and the strikethrough channel 308 and into the test garment. Fluids not immediately absorbed are held in the fluid cavity 306. The strikethrough plat may also comprise a weight ring 316 for adjusting the amount of pressure exerted by the strikethrough plate 304 on the garment.

The strikethrough plate 304 used for the present tests comprised a square acrylic plate having a thickness of 25 millimeters and a side length of 100 millimeters. The fluid cavity comprised a 25 millimeter diameter hole in the center of the plate. The hole extended 16.6 millimeters into the plate then tapered inward at a 15 degree angle, relative to the plane of the plate. The strikethrough channel 308 comprised a six-armed opening, having six 9.5 millimeter long, 1.5 millimeter wide slots arranged to extend radially from the center of the plate.

The test procedure comprised three cycles. For each cycle, 100 milliliters of simulated urine was absorbed by the test garment, and the rewet value was measured. The cycles were cumulative, so that in the second cycle the test garment had absorbed a total of 200 milliliters of simulated urine, and in the third cycle the test garment had absorbed a total of 300 milliliters of simulated urine. The steps comprising each cycle are described as follows.

The center of the absorbent core of the garment was located and marked as the insult point 302, and the strikethrough plate 304 placed on the garment over the insult point. A separatory funnel 310 was filled with 100 milliliters of simulated urine. The tip 312 of the separatory funnel 310 was placed just above the strikethrough plate. The separatory funnel 310 was opened and the flow of simulated urine was regulated using a turncock 314 to keep the fluid cavity 306 completely full of simulated urine to maintain the simulated urine at an approximately constant hydraulic pressure at the surface of the garment.

After the 100 milliliters of simulated urine was absorbed into the test garment, the strikethrough plate 304 was removed and a 0.5 psi weight (i.e., a weight having a mass that will exert 0.5 pounds per square inch on its lower surface) with a lower surface measuring 2.5 inches by 2.5 inches was placed on the insult point. After ten minutes, the 0.5 psi weight was removed and the first quantity of filter paper was placed on the insult point. the 0.5 psi weight was placed on top of the first quantity of filter paper. Ten minutes after the first quantity of filter paper was placed on the insult point, the weight and the first quantity of filter paper were removed and the first quantity of filter paper was weighed, to within one hundredth of a gram, to obtain its wet weight. The rewet value for the first insult was measured as the difference between the wet and dry weights of the first quantity of filter paper.

The above steps were repeated for the second and third cycles, using the 72 gram quantity of filter paper for the second cycle and the 90 gram quantity of tissue paper for the third cycle (larger quantities of tissue paper may be required for garments having relatively high amounts of rewet). The procedure was repeated for 30 samples of the test garment.

The procedure was then repeated for thirty samples of a conventional garment. The conventional garment was identical to the test garment, except the apertured film 18 was replaced with a conventional through air bonded carded, bicomponent fiber nonwoven, having a basis weight of 40 gsm. The results of the rewet test are provided in Table 1.

TABLE 1

| | Rewet Under Load (g) | | |
|---|---|---|---|
| | 1^{st} Insult | 2^{nd} Insult | 3^{rd} Insult |
| Absorbent Product | (100 ml total) | (200 ml total) | (300 ml total) |

| | | | |
|---|---|---|---|
| Test Garment | 0.18 g | 0.56 g | 1.94 g |
| Conventional Garment | 0.19 g | 4.03 g | 34.18 g* |

*12 samples

These data demonstrate that the present invention provides surprising and unexpected results when compared to conventional absorbent products. Specifically, the absorbent article of the invention provides significantly better rewet performance when the garment is at or near the saturation value.

### EXAMPLE 2

### Surface Wetness Test

The present invention has also demonstrated superior performance over a conventional garment in surface dryness tests, as described herein. Three test garments and three conventional garments, as described above, were tested for the surface dryness test.

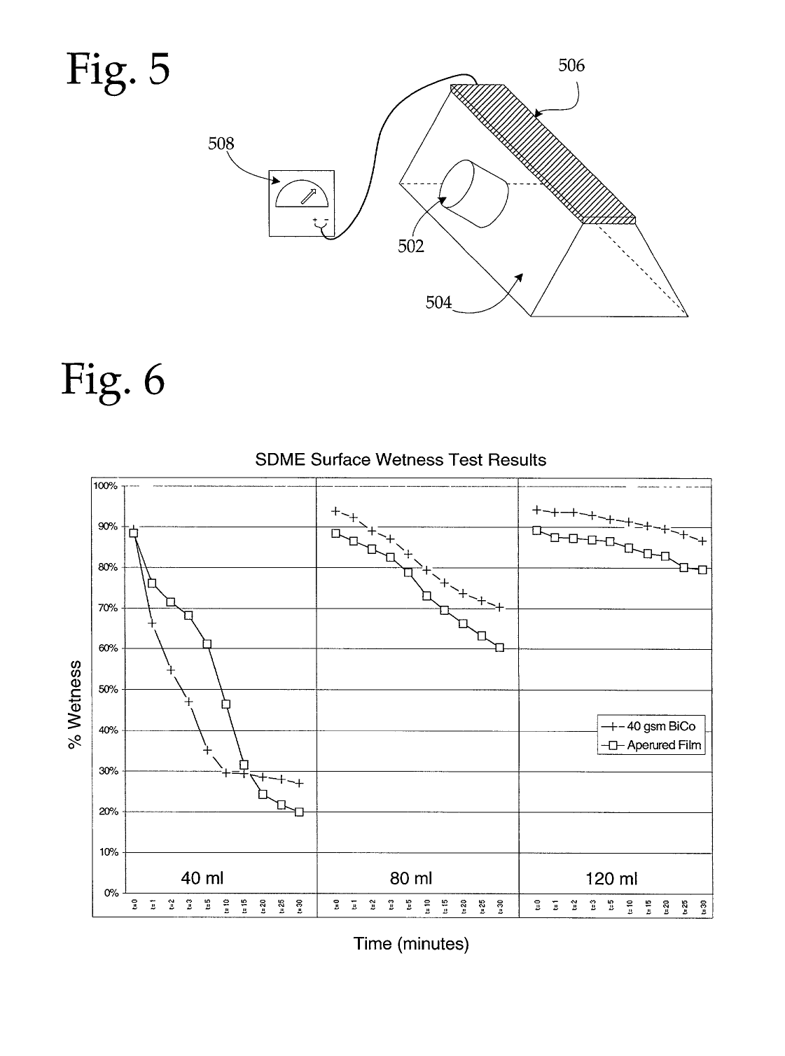

Referring now to FIG. 5, the surface dryness measuring equipment (SDME) uses an optical analysis technique to determine the surface dryness of the garment as it absorbs simulated urine. The SDME comprises a light source 502 projecting light beam into a prism 504 made of glass, crystal, or other transparent medium. A photoelectric sensor 506 is located to detect light in the prism. The light source 502 is oriented such that when the prism 504 is not in contact with any other objects, the light from the light source reflects within the prism 504, but does not strike the photoelectric sensor 506. When a droplet of fluid, such as simulated urine, contacts the prism 504, a portion of the light exits the prism 504 and enters the droplet. The light is diffused by the droplet, and some of the diffused light is returned to the prism 504. The returned light is no longer on the original path of the light beam, and may be detected by the photoelectric sensor 506. A greater number of droplets will tend to return more diffused light to the prism 504, thereby eliciting a greater response from the photoelectric sensor 506. The output of the photoelectric sensor 506 may be measured using any conventional measuring device 508, such as a chart recorder, a computer processor, a voltmeter, and the like. The SDME is used by placing a surface of the prism 504 on the topsheet 14 of the garment. As the surface wetness increases, the voltage produced by the photoelectric sensor 506 will increase. Surface dryness measuring equipment such as that described above is commercially available from Hoechst Atkiengesellschaft of West Germany. Such equipment is also described in U.S. Pat. No. 4,924,084 to Lask et al. which is incorporated herein by reference in its entirety and in a manner consistent with the present invention.

The SDME was calibrated for each type of garment (i.e., the test garment and the conventional garment). The SDME was calibrated by obtaining a reading for the unwetted sample, then completely saturating the sample and obtaining a reading for the wetness. A sample of each type of garment was stretched flat on a flat surface and the SDME was placed on the topsheet surface. The output of the SDME was recorded on a chart recorder, and the wetness reading of the dry sample was set as the 0% wetness value. Each sample was then saturated by preparing simulated urine as described elsewhere herein and pouring the simulated urine on the entire length of the garment until the garment ceased absorbing fluid. The SDME was then placed on the topsheet surface and the wetness reading of the saturated sample was set as the 100% wetness value. For the subsequent tests, the wetness readings were compared to the 0% and 100% wetness value to obtain relative percentage wetness values. Similar testing methods are disclosed, for example, in U.S. Pat. No. 5,658,268 to Johns, et al. on Aug. 19, 1997, the disclosure of which is incorporated herein by reference in its entirety and in a manner consistent with the present invention.

Each sample was tested by fixing the garment in a flattened position, marking the insult point, and locating a 70 millimeter metal ring having a 40 millimeter deep by 60 millimeter diameter circular opening therein over the insult point. The insult point for the surface dryness test samples was located about 114 millimeters (4.5 inches) from the front edge of the absorbent core. A 40 ml quantity of simulated urine was poured into the metal ring and the metal ring was removed. The SDME was then placed on the insult point and the output of the SDME was plotted for 30 minutes after being applied to the garment surface.

The above steps were repeated twice more for each garment to obtain wetness readings at 80 milliliters of load and at 120 milliliters of load. The results of these tests are shown in Table 2, and plotted in FIG. 6.

TABLE 2

| | 1^{st} Insult | | 2^{nd} Insult | | 3^{rd} Insult | |
|---|---|---|---|---|---|---|
| | (40 ml total) | | (80 ml total) | | (120 ml total) | |
| | | Test | | Test | | Test |
| | Conventional | Gar- | Conventional | Gar- | Conventional | Gar- |
| | Garment | ment | Garment | ment | Garment | ment |
| t = 0 | 89% | 88% | 94% | 88% | 94% | 89% |
| t = 1 | 66% | 76% | 92% | 86% | 94% | 87% |
| t = 2 | 55% | 72% | 89% | 84% | 94% | 87% |
| t = 3 | 47% | 68% | 87% | 82% | 93% | 87% |
| t = 5 | 35% | 61% | 83% | 79% | 92% | 86% |
| t = 10 | 30% | 46% | 79% | 73% | 91% | 85% |
| t = 15 | 29% | 32% | 76% | 70% | 90% | 84% |
| t = 20 | 29% | 24% | 74% | 66% | 90% | 83% |
| t = 25 | 28% | 22% | 72% | 63% | 88% | 80% |
| t = 30 | 27% | 20% | 70% | 60% | 87% | 80% |

The results of the surface dryness test demonstrate the surprising and unexpected result that the apertured film 18 of the present invention provides improved wetness reduction characteristics compared to a conventional transfer layer. The improvement is particularly notable with respect to the reduction in wetness for repeated void volumes, as indicated by the results for the second and third insults.

It is expected that the improved rewet under load performance of the embodiments of the present invention will be useful for reducing the rewet of absorbent articles in actual use. The reduced rewet under load may improve the comfort of an absorbent article, and may reduce the likelihood that a wearer will experience rashes, infections, and irritation caused by contact with exudates that reemerge from the absorbent core. The test results indicate that embodiments of the present invention will also provide improved performance during subsequent insults, and particularly when the garment is relatively close to its saturation point.

Although the present invention has been described in terms of particularly preferred embodiments, it is not limited to these embodiments. Alternative embodiments and modifications that would still be encompassed by the invention may be made by those skilled in the art, particularly in light of the foregoing teachings. Therefore, the following claims are intended to cover all alternative embodiments, modifications or equivalents which may be within the spirit and scope of the invention.

I claim:

1. An absorbent article comprising:
a liquid impervious outer layer;
a liquid pervious inner layer overlaying and operatively associated with the outer layer;
an absorbent core disposed between the outer layer and inner layer;
an apertured film, disposed between the inner layer and the absorbent core, comprising a liquid impervious film surface having a plurality of protrusions extending therefrom towards the absorbent core, each protrusion terminating at an aperture in the apertured film; and
wherein the absorbent article has a 200 milliliter rewet under load of less than about 1.25 grams and a 300 milliliter rewet under load of less than about 4 grams.

2. The absorbent article of claim 1, further comprising a tissue layer surrounding the absorbent core and the apertured film.

3. The absorbent article of claim 1, further comprising a transfer layer disposed between the inner layer and the absorbent core.

4. The absorbent article of claim 1, wherein the apertured film covers substantially all of a surface of the absorbent core facing the inner layer.

5. The absorbent article of claim 1, wherein the apertured film covers an insult region of the absorbent core.

6. The absorbent article of claim 1, wherein the protrusions extend in a direction substantially orthogonal to the liquid impermeable film surface.

7. The absorbent article of claim 1, wherein the protrusions are substantially circular.

8. The absorbent article of claim 1, wherein the protrusions are substantially hexagonal.

9. The absorbent article of claim 1, wherein the protrusions are substantially linear slits.

10. The absorbent article of claim 1, wherein the area of each protrusion is less at the aperture than at the film surface.

11. The absorbent article of claim 1, wherein the apertured film has a loft of between about 0.500 millimeters and about 1.500 millimeters.

12. The absorbent article of claim 1, wherein the apertured film has a loft of between about 0.750 millimeters and about 1.250 millimeters.

13. The absorbent article of claim 1, wherein the apertured film has a loft of about 1.000 millimeters.

14. The absorbent article of claim 1, wherein the apertured film has a porosity of between about 71.5 m³ₐᵢᵣ/min m²_{film} and about 122 m³ₐᵢᵣ/min m²_{film}.

15. The absorbent article of claim 1, wherein the apertured film has a porosity of between about 84.0 m³ₐᵢᵣ/min m²_{film} and about 109 m³ₐᵢᵣ/min m²_{film}.

16. The absorbent article of claim 1, wherein the apertured film has a porosity of about 96.5 m³ₐᵢᵣ/min m²_{film}.

17. The absorbent article of claim 1, wherein the apertured film has a drain rate of between about 597 kg/s m²_{film} and about 995 kg/s m²_{film}.

18. The absorbent article of claim 1, wherein the apertured film has a drain rate of between about 697 kg/s m²_{film} and about 896 kg/s m²_{film}.

19. The absorbent article of claim 1, wherein the apertured film has a drain rate of about 796 kg/s m²_{film}.

20. The absorbent article of claim 1, wherein the absorbent article has a 200 milliliter rewet under load of less than about 0.80 grams.

21. The absorbent article of claim 1, wherein the absorbent article has a 200 milliliter rewet under load of about 0.56 grams.

22. The absorbent article of claim 1, wherein the absorbent article has a 300 milliliter rewet under load of less than about 3.00 grams.

23. The absorbent article of claim 1, wherein the absorbent article has a 300 milliliter rewet under load of less than about 1.94 grams.

24. The absorbent article of claim 1, wherein the absorbent article has a surface wetness 30 minutes after a 40 milliliter insult of less than about 27%.

25. The absorbent article of claim 1, wherein the absorbent article has a surface wetness 30 minutes after an 80 milliliter insult of less than about 70%.

26. The absorbent article of claim 1, wherein the absorbent article has a surface wetness 30 minutes after a 120 milliliter insult of less than about 87%.
